(19) United States
(12) Patent Application Publication
(10) Pub. No.: US 2011/0207768 A1
(43) Pub. Date: Aug. 25, 2011

(54) PHARMACEUTICAL COMBINATION OF ANTIBACTERIAL AND ANTIFUNGAL CREAM

(75) Inventor: Michael Bornstein, Richmond, TX (US)

(73) Assignee: QUADRX PHARMACEUTICALS, Richmond, TX (US)

(21) Appl. No.: 12/722,179

(22) Filed: Mar. 11, 2010

Related U.S. Application Data
(60) Provisional application No. 61/307,719, filed on Feb. 24, 2010.

(51) Int. Cl. A61K 31/351; A61K 31/46; A61K 31/4164; A61P 31/04; A61P 31/10

(52) U.S. Cl. 514/304; 514/460; 514/399

(57) ABSTRACT

A method for the emperic treatment of an intertrigal skin infection potentially caused by a gram-positive bacteria in a human patient that includes a topical administration to the patient of a pharmaceutical formulation comprising a bactericidal amount of a Gram-positive bactericide and an antimycotic amount of an antifungal component suspended in a skin-barrier carrier.

### BACKGROUND OF INVENTION

1. Field of the Invention

Embodiments disclosed herein relate generally to methods and compositions for treating acute inflammatory skin conditions. In particular, embodiments disclosed herein relate to the combination of a bactericidal agent and an antifungal agent in the methods and compositions for treating diaper dermatitis and/or intertrigo.

2. Background Art

“Diaper rash” or diaper dermatitis is one of the most prevalent inflammatory skin conditions that afflicts infants and young children. It is generally accepted that the diaper rash is a contact irritant dermatitis that results from extended contact of the skin with urine, feces, or both. Increased wetness in the diaper area makes the skin more susceptible to damage by physical, chemical, and enzymatic mechanisms. Specifically, wet skin increases the penetration of irritant substances. Enzymes in the outermost layer of the skin can release ammonia converted from the urea present in urine, whereas enzymes in feces mixed with urine can cause an alkaline surface pH, adding to the irritation. These conditions may also allow for the growth of fungi and/or bacteria. However, most diaper dermatitis infections have historically been thought to arise from Candida albicans, a fungus found in the gastrointestinal tract. Other microbial agents have been isolated less frequently, perhaps more as a result of secondary infections. Changes in natural flora due, for example, to antibiotics prescribed for common ear and/or throat infections, may contribute to further overgrowth in the diaper areas.

Similar to diaper dermatitis, intertrigo is a skin inflammation of the body folds between adjacent areas of skin that usually develops from the chafing of warm, moist skin again skin, such in the areas of the inner thighs and genitalia, the armpits, under the breasts, the underside of the belly, behind the ears, and the web spaces between the toes and fingers. Also like diaper dermatitis, intertrigo may also be caused by either a bacterial or fungal infection that attacks the skin upon breaking of the outer layer of the skin that serves as a waterproof barrier. Such disruption of the outer layer of skin can be cause by when skin is exposed to prolonged heat, moisture, maceration, friction, lack of air circulation, and/or decreased skin pH.

Currently, compositions used to treat such skin infections target either fungal or bacterial infections, with most patients being prescribed an antifungal or bacteriostatic medication. However, the choice of one treatment or another may be incorrect because a laboratory may be required to identify the source of the infection. Thus, the patient experiences days of delay until the physician recognizes that the treatment does not work and switches to the correct treatment. Therefore, an accurate treatment necessitates the identification of an infection as either fungal or bacterial.

Although yeast and bacteria are both commonly harbored in these areas of the body, the two normally live in balance with each other and cause no symptoms. However, when the natural balance of the flora is disrupted, for example, by an anti-fungal treatment or an anti-bacterial treatment, it can diminish the growth of one microorganism and allow overgrowth of the other. For example, even when a treatment correctly treats a fungal infection, the change in natural flora that reside on the surface and with deeper layers of the skin that is now compromised may allow bacterial infections to form, further aggravating the inflammation. The reverse is also true, when a treatment correctly treats a bacterial infection, the change in natural flora in and on the compromised skin may allow fungal infections to form, further aggravating the inflammation.

Accordingly, there exists a continuing need for developments in the treatment of diaper rash and intertrigo.

### SUMMARY OF INVENTION

In one aspect, embodiments disclosed herein relate to a method for the emperic treatment of an intertrigal skin infection potentially caused by a gram-positive bacteria in a human patient that includes a topical administration to the patient of a pharmaceutical formulation comprising a bactericidal amount of a Gram-positive bactericide and an antimycotic amount of an antifungal component suspended in a skin-barrier carrier.

In another aspect, embodiments disclosed herein relate to a pharmaceutical formulation for the empiric treatment of an intertrigal skin infection potentially caused by a Gram-positive bacteria that includes a bactericidal amount of a Gram-positive bactericide and an antimycotic amount of an antifungal component suspended in a skin-barrier carrier.

Other aspects and advantages of the invention will be apparent from the following description and the appended claims.

### DETAILED DESCRIPTION

Embodiments disclosed herein relate generally to methods and compositions for treating acute inflammatory skin conditions. In particular, embodiments disclosed herein relate to the combination of a bactericidal agent and an antifungal agent in the methods and compositions for treating diaper dermatitis and/or intertrigo.

The compositions of the present disclosure, in particular the combination of a bactericidal agent and an antifungal agent may allow for the empiric treatment of diaper dermatitis and/or intertrigo. As known in the art, an empiric treatment refers to the initiation of treatment prior to determination of a firm diagnosis as an initiative against an anticipated and likely cause of an infectious disease. The empiric treatment of the present disclosure may be broad-spectrum, in that it may treat a wide variety of possible microorganisms, including both bacteria and fungus. Use of such a broad-spectrum empiric treatment may allow for a more speedy recovery by a patient and avoid the need for laboratory tests. Additionally, the treatment may also prevent the growth of other microorganisms that did not originally contribute to the skin infection.

Additionally, while diaper dermatitis has conventionally been though to result from fungal infections, the inventor of the present application has observed an increased incidence of bacterial infections in diaper dermatitis. Most of these bacterial infections are caused by bacteria belonging to the Streptococcus genus or Staphylococcus genus, both of which are Gram-positive bacteria, including infections due to Methicillin-resistant Staphylococcus aureus (MRSA), which is a strain of a staph bacterium that has become resistant to many antibiotics, in diaper dermatitis. Accordingly, the treatment compositions and methods of the present disclosure may allow for the treatment of a bacterial infection, including MRSA infections.

In particular, the compositions of the present disclosure may include at least three components: a bactericidal agent, an antifungal agent, and a skin-barrier carrier. The presence of each of these components may allow for an empiric treatment for any fungal or bacterial infection while also providing the necessary skin barrier properties to prevent the susceptible under skin layers from further attack.

Generally, anti-bacterial agents may be classified into two types, bactericides and bacteriostatics. Bactericidal antibiotics kill bacteria whereas bacteriostatic antibiotics only slow their growth or reproduction. The inventor of the present application has found that the use of bacteriostatics in a treatment does not provide a broad-spectrum empiric treatment, particularly in view of the inventor's increased clinical observation of infections in diaper dermatitis and intertrigo resulting from MRSA.

In a particular embodiment, the treatment composition of the present disclosure may include a Gram-positive bactericide. As used herein, a Gram-positive bactericide refers to an antibacterial agent that kills at least some Gram-positive bacteria. It would be understood by those skilled in the art that a particular anti-bacterial agent may have different effectiveness on different bacteria, and that while a Gram-positive bactericide may also kill some Gram-negative bacteria, it possess broader bactericidal activity against Gram-positive bacteria than Gram-negative bacteria. In certain embodiments, the Gram-positive bactericide may possess bactericidal activity against Streptococcus and/or Staphylococcus bacteria, including most strains of Staphylococcus aureus, Staphylococcus epidermidis, Staphylococcus saprophyticus, and Streptococcus pyogenes. In a more particular embodiment, the Gram-positive bactericide may possess bactericidal activity against methicillin-resistant Staphylococcus aureus and group A beta-hemolytic streptococci.

In a more particular embodiment, the Gram-positive bactericide may include one or more pseudomonic acids, salts thereof, or esters thereof. Acceptable salts may include alkali metal or alkaline earth metal salts, and esters may include C1 to C4 alkyl esters. Pseudomonic acids are fermentation products of the bacteria Pseudomonas fluorescens. These biosynthesis products include four acids, pseudomonic acid A-D, but generally, pseudomonic acid A possesses the greatest bactericidal activity. Therefore, in a particular embodiment, the Gram-positive bactericide may include pseudomonic acid A, but may also include one or more other pseudomonic acids (pseudomonic acid B-D). Pseudomonic acid A may comprise at least 90% of the bactericidal component in more particular embodiments. Pseudomonic acid A is shown by the following formula:

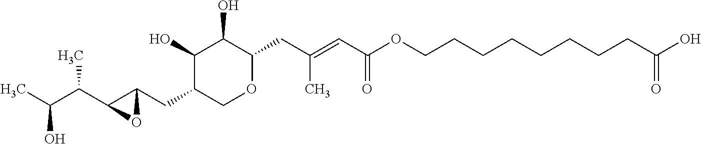

which is also known by the name mupirocin. Pseudomonic acid B possesses an additional hydroxyl group at C8; pseudomonic acid C possesses a double bond between C10 and C11, instead of the epoxide; and pseudomonic acid D possesses a double bond at C4′ and C5′ in the 9-hydroxy-nonanoic acid portion the compound.

Alternatively, the Gram-positive bactericide may be a pleuromutilin antibiotic such as retapamulin. Pleuromutilin, a naturally occurring antibiotic produced by Pleurotus mutilus and P. passeckerianus has the general formula of:

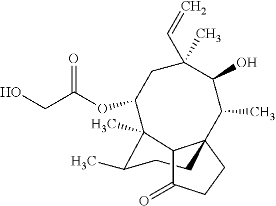

whereas derivatives of the naturally occurring pleuromutilin that have the principal ring structure of pleuromutilin and also possess antibacterial activity have also been developed. Many of such derivatives, including retapamulin, vary in the chain that extends as the carboxylate group.

An “bactericidal amount” of a given bactericide means an amount at which the topically active bactericide component kills bacteria associated with infections. The Gram-positive bactericides may be present in the compositions of the present disclosure in a bactericidal amount that may range from about 0.01% to 10% by total weight of the treatment composition in a particular embodiment. In a more particular embodiment, the Gram-positive bactericides may be present in the composition in an amount compound is 0.25 to 3% by total weight of the treatment composition.

In addition to a bactericide, the treatment composition of the present disclosure also includes at least one antifungal component. Examples of the antifungal components may include miconazole, ketoconazole, econazole, terbinafine, ciclopirox, tolnaftate, sertaconazole, sulconazole, amphotericin b, cholorxylenol, clioquinol, butenafine, naftifine, nystatin, and clotrimazole. An “antimycotic amount” of a given antifungal component means an amount at which the antifungal composition component hinders the growth of fungus associated with infections. The antifungal components may be present in the formulations of the present disclosure in an antimycotic amount that may range from about 0.01% to 10% by total weight of the treatment composition in a particular embodiment, and from 0.25% to 2% by total weight in a more particular embodiment.

Additionally, the bactericide and antifungal components are provided in a skin-barrier carrier. As used herein, the term “skin-barrier carrier” refers to a carrier mechanism for topical application of that is capable of forming a protective skin barrier against wetness on the infected skin. In particular, because the infection causing the diaper dermatitis and/or intertrigo has disrupted the outermost skin layer which generally serves as a protective layer to the under layers of skin, the skin-barrier properties of the carriers of the present disclosure may minimize or reduce further irritation or damage to the skin by contact irritants (including wetness, urine, feces, ammonia, changes in pH, friction, etc.) as well as deliver the treatment components to the infection. Examples of such skin-barrier carriers include petrolatum, bees wax, lanolin, balsam, and paraffin, and may optionally also include any of zinc oxide, mineral oil, ceresin, and dimethicone.

In a particular embodiment of the present disclosure, the composition comprises the following combination all suspended in white petrolatum: 0.25% to 2% miconazole by weight, 0.3% to 2% mupirocin by weight, and 10% to 40% by weight zinc oxide.

Embodiments of the present disclosure may provide at least one of the following advantages. The formulations of the present disclosure may be used to treat diaper dermatitis and/or intertrigo with an empiric therapy. Specifically, a single treatment may be prescribed without a definitive diagnosis of the cause of the infection. Additionally, with the increasing occurrence of MRSA, the formulation may be effective in treating the drug-resistant strains. Further, when the natural balance of the flora is disrupted, for example, by an anti-fungal treatment or an anti-bacterial treatment, it can diminish the growth of one microorganism and allow overgrowth of the other. Therefore, the formulations of the present disclosure may prevent such secondary infections from occurring.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed:

1. A method for the empiric treatment of an intertrigal skin infection potentially caused by a gram-positive bacteria in a human patient, comprising:
a topical administration to the patient of a pharmaceutical formulation comprising a bactericidal amount of a Gram-positive bactericide and an antimycotic amount of an antifungal component suspended in a skin-barrier carrier.

2. The method of claim 1, wherein the Gram-positive bactericide has bactericidal activity against Streptococcus and/or Staphylococcus bacteria.

3. The method of claim 2, wherein the Gram-positive bactericide has bactericidal activity against methicillin-resistant Staphylococcus aureus.

4. The method of claim 1, wherein the Gram-positive bactericide comprises pseudomonic acid A, salts thereof, or esters thereof.

5. The method of claim 1, wherein the Gram-positive bactericide comprises a pleuromutilin derivative.

6. The method of claim 1, wherein the pleuromutilin derivative is retapamulin.

7. The method of claim 1, wherein the bactericidal amount ranges from about 0.01% to 10% by total weight of the treatment composition.

8. The method of claim 7, wherein the bactericidal amount ranges from about 0.25 to 3% by total weight of the treatment composition.

9. The method of claim 1, wherein the antifungal comprises at least one of miconazole, ketoconazole, econazole, terbinafine, ciclopirox, tolnaftate, sertaconazole, sulconazole, amphotericin b, cholorxylenol, clioquinol, butenafine, naftifine, nystatin, or clotrimazole.

10. The method of claim 1, wherein the antimycotic amount ranges from about 0.01% to 10% by total weight of the treatment composition.

11. The method of claim 10, wherein the antimycotic amount ranges from about 0.25% to 2% by total weight of the treatment composition.

12. The method of claim 1, wherein the skin-barrier carrier comprises at least one of petrolatum, bees wax, lanolin, balsam, and paraffin.

13. The method of claim 1, wherein the pharmaceutical formulation further comprises at least one of zinc oxide, mineral oil, ceresin, or dimethicone.

14. The method of claim 1, wherein the pharmaceutical formulation comprises 0.25% to 2% miconazole by weight, 0.3% to 2% mupirocin by weight, and 10% to 40% by weight zinc oxide in petrolatum.

15. A pharmaceutical formulation for the empiric treatment of an intertrigal skin infection potentially caused by a Gram-positive bacteria, comprising:
a bactericidal amount of a Gram-positive bactericide and an antimycotic amount of an antifungal component suspended in a skin-barrier carrier.

16. The formulation of claim 15, wherein the Gram-positive bactericide has bactericidal activity against Streptococcus and/or Staphylococcus bacteria.

17. The formulation of claim 16, wherein the Gram-positive bactericide has bactericidal activity against methicillin-resistant Staphylococcus aureus.

18. The formulation of claim 15, wherein the Gram-positive bactericide comprises pseudomonic acid A, salts thereof, or esters thereof.

19. The formulation of claim 15, wherein the Gram-positive bactericide comprises a pleuromutilin derivative.

20. The formulation of claim 15, wherein the pleuromutilin derivative is retapamulin.

21. The formulation of claim 15, wherein the bactericidal amount ranges from about 0.01% to 10% by total weight of the treatment composition.

22. The formulation of claim 21, wherein the bactericidal amount ranges from about 0.25 to 3% by total weight of the treatment composition.

23. The formulation of claim 15, wherein the antifungal comprises at least one of miconazole, ketoconazole, econazole, terbinafine, ciclopirox, tolnaftate, sertaconazole, sulconazole, amphotericin b, cholorxylenol, clioquinol, butenafine, naftifine, nystatin, or clotrimazole.

24. The formulation of claim 15, wherein the antimycotic amount ranges from about 0.01% to 10% by total weight of the treatment composition.

25. The formulation of claim 24, wherein the antimycotic amount ranges from about 0.25% to 2% by total weight of the treatment composition.

26. The formulation of claim 15, wherein the skin-barrier carrier comprises at least one of petrolatum, bees wax, lanolin, balsam, and paraffin.

27. The formulation of claim 15, wherein the pharmaceutical formulation further comprises at least one of zinc oxide, mineral oil, ceresin, or dimethicone.

28. The formulation of claim 15, comprising 0.25% to 2% miconazole by weight, 0.3% to 2% mupirocin by weight, and 10% to 40% by weight zinc oxide in petrolatum.
